(19) United States
(12) Patent Application Publication
(10) Pub. No.: US 2023/0277049 A1
(43) Pub. Date: Sep. 7, 2023

(54) Endoscope Systems with Detachable Scopes

(71) Applicant: The Board of Trustees of the Leland Stanford Junior Univerity, Stanford, CA (US)

(72) Inventor: Anna Sophie von Wendorff, Loomis, CA (US)

(73) Assignee: The Board of Trustees of the Leland Stanford Junior University, Stanford, CA (US)

(21) Appl. No.: 18/179,066

(22) Filed: Mar. 6, 2023

Related U.S. Application Data
(60) Provisional application No. 63/316,719, filed on Mar. 4, 2022.

(51) Int. Cl. A61B 1/05; A61B 1/06

(52) U.S. Cl. CPC A61B 1/053; A61B 1/051; A61B 1/0669; A61B 1/0684

(57) ABSTRACT

Endoscope systems with detachable scopes in accordance with embodiments of the invention are disclosed. In one embodiment of the invention, an endoscope system comprises an endoscope body and a detachable scope. The endoscope body comprises a cable capable of transmitting image data to a computing system, and a body connector. The detachable scope comprises a scope connector configured to couple with the body connector, and a tip, comprising an illumination module and an image sensor, wherein image data produced by the image sensor is transmitted to the endoscope body via the scope connector.

### STATEMENT OF FEDERALLY SPONSORED RESEARCH

This invention was made with Government support under 2133585 awarded by the National Science Foundation. The Government has certain rights in the invention.

### CROSS REFERENCE TO RELATED APPLICATION(S)

The current application claims priority to U.S. Provisional Pat. Application Serial No. 63/316,719 entitled “USB Endoscope,” filed Mar. 4, 2022, the disclosure of which is herein incorporated by reference in its entirety.

### FIELD OF THE INVENTION

This invention generally relates to endoscopes for use in diagnostic, therapeutic, and surgical applications. More particularly, this invention relates to an endoscope or endoscopy system that includes a body with a removable scope.

### BACKGROUND

Endoscopes are minimally invasive medical devices used to examine the internal anatomy of the patient. A medical professional may use an endoscope to investigate symptoms, confirm a diagnosis, and/or provide treatment. An endoscope is an instrument with a rigid or flexible tube, a lighting system to illuminate the organ or anatomical region, and an imaging system to transmit images to the viewer. Various types of endoscopes are available for examination of different organs, such as a cystoscope for the lower urinary tract, an enteroscope for the small intestine, a bronchoscope for the lower respiratory tract, and many others. The endoscope is typically inserted directly into the organ or anatomical region, and may be fitted with a further apparatus for examination or retrieval of tissue. Modern endoscopes are often videoscopes, transmitting images from a camera to a screen for real-time viewing by the health professional. The procedure may then be reviewed through video playback, or condensed into a few still images with notes and drawings.

Nasoendoscopes, also called “rhinoscopes, “sinusscopes,” “nasolaryngoscopes”, or “nasal endoscopes,” are a category of endoscopes which examine the nasal passage, sinonasal areas, and further posterior sections of the airway. A nasoendoscopy is a common procedure used for the diagnosis and management of nasal, sinus, and resonance conditions including but not limited to nasal polyps, velopharngeal dysfunction, and nasal and paranasal sinus cancers. A nasoendoscope provides superior and complementary visualization capabilities of the nasal passage, soft palate, and throat when compared to other nasal examination techniques such as Magnetic Resonance Imaging, computed tomography, fluoroscopy, nasometry, and anterior rhinoscopy with nasal speculum.

Because nasoendoscopes are small in diameter (generally less than 5.0 mm in diameter), they can pass through the nasal cavity when other endoscopes cannot reach this area. Nasoendoscopes generally consist of a thin axis filled with optics, fiberoptics, or electronic sensors which transmit light into the internal anatomy out of the patient, and capture images of areas of interest.

### SUMMARY OF THE INVENTION

Portable endoscope systems with detachable scopes, in accordance with various embodiments of the invention, are disclosed.

In one embodiment of the invention, an endoscope system comprises an endoscope body, comprising a cable capable of transmitting image data to a computing system; and a body connector; and a detachable scope, comprising a scope connector configured to couple with the body connector; and a tip, comprising an image sensor, wherein image data produced by the image sensor is transmitted to the endoscope body via the scope connector; and an illumination module capable of providing light.

In a further embodiment, the illumination module is physically separated from the image sensor such that the image sensor is protected from heat generated by operation of the illumination module.

In another embodiment, the image sensor is coupled with an image sensor printed circuit board (PCB) and the illumination module includes an illumination printed circuit board (PCB), and wherein the tip has a diameter of less than 3 mm.

In a yet further embodiment, the detachable scope is configured to be resistant to liquid in its entirety such that when detached from the endoscope body, the detachable scope can be immersed in its entirety in liquid disinfectant while maintaining suitability for use in an endoscopy procedure subsequent to the immersion.

In another embodiment, the endoscope body further comprises a main circuitry coupled with the cable for receiving power from the computing system.

In yet another embodiment, the endoscope body further comprises an image processor coupled with the body connector to receive the image data, wherein the image processor is capable of processing the image data and transmitting the processed image data to the computing system.

In still another embodiment, the cable is a Universal Serial Bus (USB) cable.

In a still further embodiment, the computing system includes at least one device selected from the group consisting of a personal computer, a monitor, a tablet and a smartphone.

A detachable scope for an endoscope system, in a yet further embodiment of the invention, comprises a scope connector configured to couple with a body connector of an endoscope body; and a tip, comprising an illumination module capable of providing light; and an image sensor, wherein image data produced by the image sensor is transmitted to the endoscope body via the body connector.

In yet another embodiment, the illumination module is physically separated from the image sensor such that the image sensor is protected from heat generated by operation of the illumination module.

In another further embodiment, the image sensor is coupled with an image sensor printed circuit board (PCB) and the illumination module includes an illumination printed circuit board (PCB), and wherein the tip has a diameter of less than 3 mm.

In still another further embodiment, the detachable scope is configured to be resistant to liquid in its entirety such that when detached from the endoscope body, the detachable scope can be immersed in its entirety in liquid disinfectant while maintaining suitability for use in an endoscopy procedure subsequent to the immersion.

In a still yet further embodiment, the detachable scope is of an elongated shape and is coupled to the endoscope body such that a proximal end of the elongated shape is disposed near the endoscope body and a distal end of the elongated shape extends away from the endoscope body.

An endoscope body for an endoscope system, in still yet another embodiment of the invention, comprises a cable capable of transmitting image data to a computing system; and a body connector configured to couple with a scope connector of a detachable scope, wherein image data produced by an image sensor of the detachable scope is received from the detachable scope via the body connector.

In a still further embodiment again, the endoscope body further comprises a main circuitry coupled with the cable for receiving power from the computing system.

In still another embodiment again, the endoscope body further comprises an image processor coupled with the body connector to receive the image data, wherein the image processor is capable of processing the image data and transmitting the processed image data to the computing system.

In a yet further embodiment, the cable is a Universal Serial Bus (USB) cable.

In another further embodiment, the computing system includes at least one device selected from the group consisting of a personal computer, a monitor, a tablet and a smartphone.

In still another embodiment, at least one portion of the endoscope body is formed in the shape of a handle.

An endoscope system, in still yet another embodiment of the invention, comprises an endoscope body, comprising a cable capable of transmitting image data to a computing system, wherein upon coupling the cable with the computing system, the endoscope system is automatically recognized as a webcam by the computing system; and a body connector; and a detachable scope, comprising a scope connector configured to couple with the body connector; and a tip, comprising an image sensor coupled with an image sensor printed circuit board (PCB), wherein image data produced by the image sensor is transmitted to the endoscope body via the scope connector; and an illumination module including an illumination printed circuit board (PCB) coupled with three light-emitting diodes (LEDs),wherein the illumination module is physically separated from the image sensor such that the image sensor is protected from heat generated by operation of the illumination module, wherein the image sensor PCB and the illumination PCB are configured so as to enable the tip to have a diameter of less than 3 mm, and wherein the detachable scope is configured to be resistant to liquid in its entirety such that when detached from the endoscope body, the detachable scope can be immersed in its entirety in liquid disinfectant while maintaining suitability for use in an endoscopy procedure subsequent to the immersion.

### BRIEF DESCRIPTION OF THE DRAWINGS

FIG. 1 illustrates an endoscope system in accordance with certain embodiments of the invention.

FIG. 2 illustrates scopes in sterile packaging in accordance with certain embodiments of the invention.

FIGS. 3A-3B illustrate various views of a scope tip assembly of a scope in an endoscope system in accordance with certain embodiments of the invention.

FIG. 3C illustrates a scope tip assembly in accordance with certain embodiments of the invention.

FIG. 4 illustrates a cross-sectional view of a scope tip of a scope with two image sensors in accordance with certain embodiments of the invention.

FIG. 5 illustrates an overhead view of an endoscope system in accordance with certain embodiments of the invention.

FIG. 6 is a diagram of an endoscope system connected with a computing system in accordance with certain embodiments of the invention.

The figures provide examples in accordance with various embodiments of the invention, and are not intended to limit the scope of the invention. In addition, the figures may not necessarily be drawn to scale. It is understood that various aspects illustrated in the figures may be modified, combined, omitted without departing from the scope of the invention.

### DETAILED DESCRIPTION

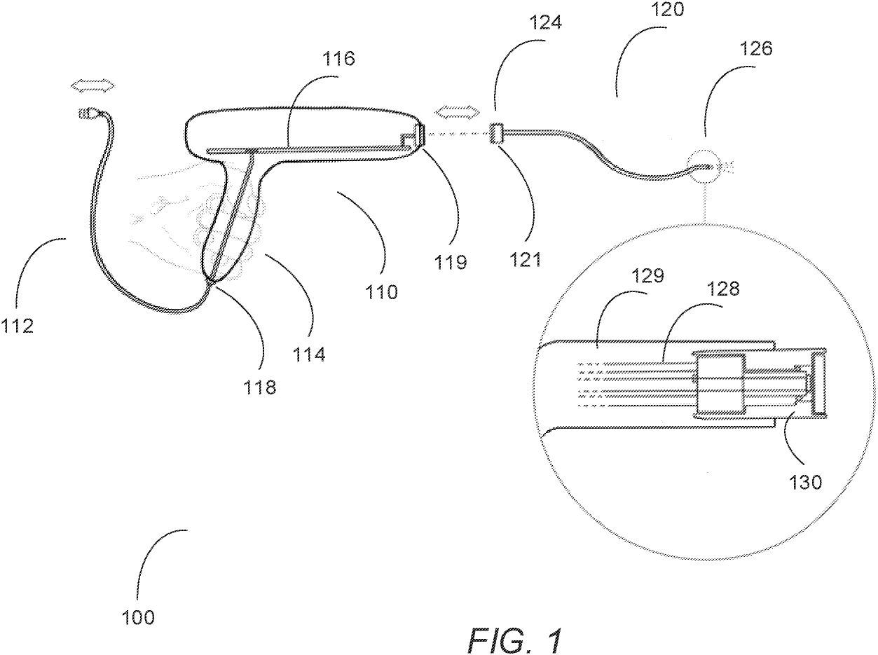

Turning now to the drawings, endoscope systems, or endoscopes, with detachable scopes are illustrated. In many embodiments of the invention, an endoscope system 100 as shown in FIG. 1 is portable and functions without the need for a surgical tower or any proprietary equipment. The scope 120 portion of the system is detachable from the endoscope body 110, allowing for a different scope that is new or fully disinfected to be attached to the endoscope body for each patient. Further, in certain embodiments of the invention, the system can record the endoscopy examination for future reference. Endoscope systems can be configured for use in the examination of one or more of various organs or anatomical regions of the body.

In a number of embodiments of the invention, the endoscope system is configured to be used as a nasoendoscope for examination of the nasal passage and neighboring anatomy. In the past, certain nasoendoscopes achieved visualization with a series of lenses or glass rods encased in a metal tube. Such a “fiberscope,” “fiberoptic endoscope,” or “rigid endoscope” often has a number of drawbacks: they are fragile, have low resolution, require additional lighting units, and/or the procedure cannot be recorded. Users were usually required to observe in real-time the anatomy of the patient. A recording of the endoscopy often proved to be important for assessing changes over time, for liability and reimbursement purposes, and for consultations with other medical professionals. Some attempted solutions tried to attach sensors onto the eyepiece of the nasoendoscope, but this often led to high manufacturing expenses and rendered the device difficult to maneuver.

More recently, “digital” or “chip-in-tip” or “flexible” nasoendoscopes appeared on the market. These pass image sensors (commonly CCD or CMOS) into the nostril of the patient, and the image is electronically relayed back to a proprietary device or surgical tower. These “chip-in-tip” nasoendoscope systems also typically have a number of drawbacks: they are expensive, difficult to disinfect, heavy and difficult to hold, often have complex therapeutic additions, and require additional specialty equipment (such as a surgical tower) that is large and non-portable. Some attempted solutions have allowed for recording the procedure and then placing the recorded data onto a dedicated processor unit, but this often rendered the file difficult to access for industry professionals.

Further, “fiberoptic” as well as “chip-in-tip” nasoendoscopes require additional equipment (commonly called a surgical tower) in order to conduct the nasoendoscopy. This commonly includes lighting, power, and/or monitor equipment on a cart. Not only does the requirement of a surgical tower render this type of system non-portable, but the multiple cables often hinder the user’s movement. Attempted solutions have employed wireless data transmission, relying heavily on battery capabilities which are by nature limited and increase the weight of the device. In addition, difficulties in maneuvering the surgical tower often result in doctors having to look away from the patient during the procedure, complicating the procedure and increasing risk of injury.

Current scopes and their patient-contacting scope portions are usually reprocessed manually. In this process, they are typically immersed in high-level liquid disinfectant for a specified period time, such as 13-33 minutes. Current diagnostic endoscope handles cannot withstand immersion. As a result, endoscopes are placed half-in, half-out of the disinfectant, which increases the risk of accidental infection between patients. High-resource institutions may be able to purchase endoscope reprocessing machines, but this can be a significant cost for many clinics. Autoclave machines have a lengthy processing time, requiring users to purchase multiple endoscope units to account for the down-time. Protective barriers (such as EndoSheath) are a single-use option for use with endoscopes, but have been known to tear, thus being rendered ineffective.

Previously existing endoscope systems present a number of additional issues. They may be difficult to hold without a specified grip. Users of many nasoendoscopes, for example, grip the outer diameter of the device which is not comfortable for a wide range of users, especially users with small hands. Many systems can be heavy, leading to long-term musculoskeletal pains such as in the back and spine for the user. Certain nasoendoscopes include features such as jointed tips that can be articulated, or working channels for suction, irrigation, or other uses. These features add complexity to the product, resulting in higher chance of breakage, increased difficulty in disinfection, and increased manufacturing costs. Patient deaths have resulted from improperly reprocessed endoscopes that resulted in cross contamination between patients. Further, many endoscopes such as but not limited to nasoendoscopes appear frightening to patients as a result of several features including their color, shape and size. This can result in non-compliance, trauma for the patient, and unnecessary physician time.

Embodiments of the invention provide endoscope systems that are inexpensive, portable, easily disinfectable and/or employ disposable parts, and/or that do not require the use of a separate, specialized surgical tower.

### Endoscope Systems

Endoscope systems according to various embodiments of the invention are configured to run independently of any surgical tower or proprietary equipment. As an example, endoscope systems such as endoscope system 100 may be powered using a source of power such as but not limited to a cable 112, power outlet, and/or a battery. Being implemented independently of an unwieldy surgical tower, the endoscope system may be easily maneuvered and moved around a patient room or between hospital buildings. A display or monitor for viewing the endoscopy examination may also be positioned as desired. For example, the monitor may be placed behind the patient so as to allow the physician to view the monitor along his/her line of sight, thus improving hand-eye coordination. In many embodiments of the invention, any monitor can be used, reducing duplicate equipment as most clinic rooms already have monitors installed. In addition, a smaller overall system size can allow for lower shipping costs for sales and repair purposes, and removing the need for a surgical tower also enables potentially significant cost savings.

In some embodiments of the invention, the cable is a USB cable that connects the endoscope system to a computing system, including (but is not limited to) one or more of a variety of computing devices, such as a computer, monitor, tablet, and/or smartphone. In a number of embodiments of the invention, existing software in a computing system can recognize the endoscope system as a device, such as but not limited to a camera device. The computing system can employ existing drivers or install appropriate drivers to communicate with the endoscope system. This allows the endoscope system to be implemented as a plug-and-play device recognizable by conventional computing devices and/or other off-the-shelf electronic equipment, simplifying the integration of the endoscope system into a clinic’s practice, regardless of its pre-existing electronic medical record system. During an endoscopy procedure, image data of the examination can be transmitted to the computing system and displayed on a monitor. In certain embodiments of the invention, user input at the computing system can control various components of the endoscope system.

In some embodiments of the invention, data from endoscope system is transmitted via a communication channel, such as but not limited to a USB cable and/or a wireless connection. In certain embodiments, a single USB cable connected to a computing system may be used both for data transmission and as a power source for the endoscope system. The endoscope system and/or the computing system it is connected to according to many embodiments of the invention allows the user to view, record, and/or share the endoscopy examination video. This can be implemented by, for example and not by way of limitation, incorporating a standard video streaming functionality such as but not limited to USB Video Class (UVC). The examination recording can be saved on a connected computing system in one or more of a variety of formats, such as but not limited to YUV, MJPEG, MPEG-2 TS, H.264, and/or DV. Employing a streaming standard can allow for a simple and efficient setup of the endoscope system for saving and organizing recorded procedure videos. Consistent recording of endoscopies can improve long-term care for the patient as changes can be observed over time. In addition, endoscopy data files can be shared with other specialists, aiding in effectiveness of in-person referrals and increasing treatment success.

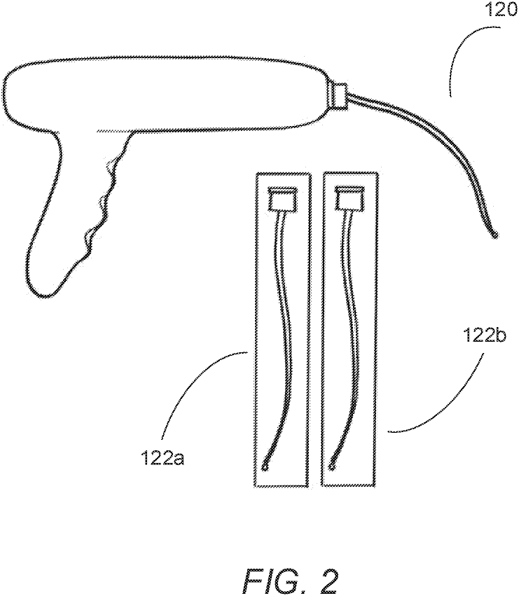

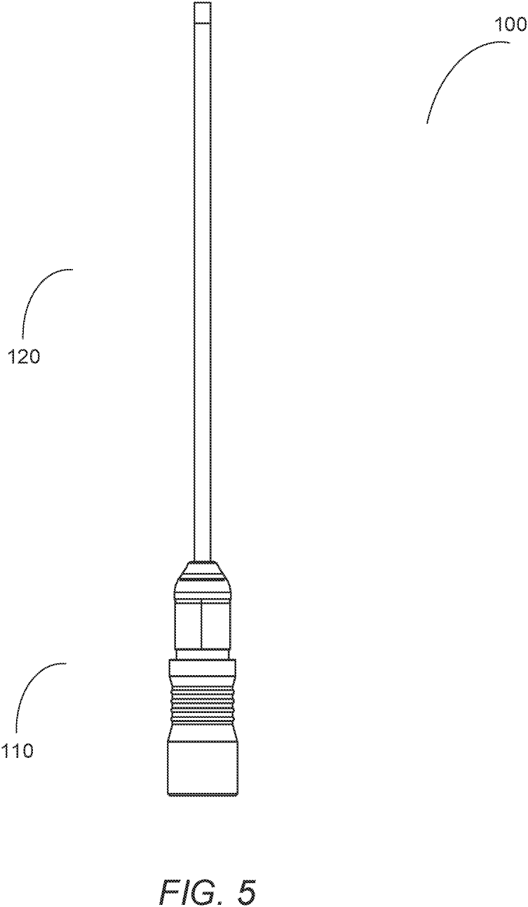

According to many embodiments of the invention, a scope such as scope 120 within the endoscope system comes into contact with the patient and is completely detachable from an endoscope body such as endoscope body 110 of the endoscope system. The scope can withstand complete immersion in high-level disinfectant liquid. This allows for fewer reprocessing failures from inadequate disinfection, as well as less down-time between patients, as the scope can be removed and replaced by a clean scope immediately. Removable scopes can also be delivered sterile to users, in sterile packaging 122a-b (such as that shown in FIG. 2) according to ISO guidelines/standards, saving reprocessing time and decreasing risk of infection. In some embodiments of the invention, practitioners can dispose of the scope portion of the endoscope system after each use and replace it with a new scope entirely. In many embodiments of the invention, the endoscope body is submersible and can withstand complete immersion in high-level disinfectant liquid. An overhead view of an endoscope system in accordance with some embodiments of the invention is shown in FIG. 5.

While various endoscope systems are described above with respect to FIGS. 1-2, other endoscope systems may be utilized as appropriate to the requirements of a specific application in accordance with various embodiments of the invention. Bodies of endoscope systems in accordance with a number of embodiments of the invention are discussed further below.

### Endoscope Bodies

As shown in FIG. 1, an endoscope body, such as endoscope body 110, of an endoscope system such as endoscope system 100 according to some embodiments of the invention is configured for effective handling during an endoscopy procedure. The endoscope body may be configured to be held by hand, and may include a handle portion such as handle portion 114 formed in the shape of a handle or other appropriate shape. The handle portion may include a grip, or may include other mechanisms for attachment to one or more of various portions of the user’s or patient’s body such as but not limited to a wristband or clip. The endoscope body in accordance with other embodiments of the invention is configured to be otherwise positioned and/or stabilized such that the practitioner can maneuver the endoscope system effectively. The endoscope body can be made with any suitable material such as but not limited to polyethylene, polypropylene, thermoplastic polyurethane, and acrylonitrile butadiene styrene.

In some embodiments of the invention, disposed within the endoscope body is a main circuitry, such as main circuitry 116, which may be coupled with one or more of a variety of mechanisms for powering and/or transmitting data to and/or from the endoscope system, such as but not limited to the cable 112. In some embodiments of the invention, the cable connects to the main circuitry within the endoscope body, and exits through a cable pass-through, or opening, 118. The opening may be waterproof or water resistant, and can be coupled with a cable gland or strain relief. Alternatively, the endoscope body may include one or more connection mechanisms configured to receive one or more sources for power and/or data transmission, such as but not limited to a USB cable, power cord or battery. For example, an electrical connection mechanism may transfer power from a computing system to the main circuitry. An electrical connection mechanism can also provide power to components in the detachably coupled scope 120, either directly or via another component such as but not limited to the main circuitry. As another example, the main circuitry may transfer image data through a communication channel to the computing system. The main circuitry may include, for example, a printed circuit board (PCB).

The main circuitry can be coupled with a body connector, such as body connector 119, which can connect with a scope connector to establish an electrical communication between the endoscope body and a detachable scope. The connectors may include or be coupled with one or more of various types of electrical connection mechanisms, such as but not limited to wires or other connection mechanisms capable of transferring power and/or data between components of the endoscope body and scope.

In many embodiments of the invention, the endoscope body includes an image processor, which may be coupled with, for example, the main circuitry and/or the cable. The image processor may be capable of performing one or more of a variety of image processing tasks. These tasks can include (but are not limited to) processing the analog image signal into a digital image signal, such as but not limited to a UVC signal, and/or calculating the shape, size or other characteristics of anatomical structures and openings through, for example, the use of color pixelation.

In certain embodiments of the invention, the endoscope body comprises an elongated chamber that may hold liquid such as a disinfectant. The chamber may be formed externally and/or separately from the handle portion, or internally as an indentation within the handle portion. As an example and not by way of limitation, the chamber may extend along at least part of the length of the handle portion, such as vertically along the handle shape of the handle portion. The scope can be detached from the endoscope body and placed in the chamber for various purposes such as but not limited to storage, disinfection and/or calibration. In several embodiments of the invention, a calibration target such as but not limited to the USAF resolution test chart may be positioned at the outer end of the chamber.

In some embodiments of the invention, the endoscope body further comprises a slot for the insertion of a memory device configured to store data for subsequent access. Some endoscope bodies include a fixed or adjustable display for displaying image data.

The endoscope body can be configured such that during patient examination, it does not come in contact with the patient. In many embodiments of the invention, the endoscope body is configured to be water-resistant and/or suitable for cleaning, such as but not limited to via wiping with a disinfectant wipe. In some embodiments of the invention, the endoscope body is configured to be suitable for immersion in liquid in its entirety, without compromising its effectiveness or safety in performing endoscopy procedures. The endoscope body may be configured such that it can be placed in chemical disinfectant for a particular minimum amount of time deemed sufficient for proper disinfection. As an example, the body connector, cable connection portion, and/or cable plug can be sealed so as to allow power and data signals to pass through, but not allow liquids or potentially contaminating substances to enter the endoscope body. This can be done by using potting methods such as epoxy placed around the connecting pins within the connector subassembly. It can also be done by bonding or welding ring-shaped materials around each pin. Alternatively or additionally, the endoscope body may be sterilized or disinfected using methods such as but not limited to EtO sterilization, Gamma sterilization, or high-level disinfectant immersion. The endoscope body may include components that are resilient and/or hardened against radiation, so as to withstand certain sterilization methods.

While various endoscope bodies are described above with respect to FIG. 1, other endoscope bodies may be utilized as appropriate to the requirements of a specific application in accordance with various embodiments of the invention. Detachable scopes in accordance with a number of embodiments of the invention are discussed further below.

### Detachable Scopes

In many embodiments of the invention, the scope is removably attached to the endoscope body via a connection between a scope connector such as scope connector 121 and a body connector such as body connector 119. In a number of embodiments of the invention, the scope is of an elongated form with a proximal end 124 (proximal to the endoscope body when attached) and a distal end 126 opposite the proximal end 124. The scope connector may be disposed at the proximal end of the scope, and a scope tip such as scope tip 130 at the distal end.

In accordance with many embodiments of the invention, prior to performing an endoscopy, a medical practitioner connects a sterile scope to the endoscope body. At least a portion of the scope, such as but not limited to the scope tip, is inserted into the patient during an endoscopy so as to bring the scope tip in proximity to the internal anatomical area of interest. Following the procedure, the scope is disconnected from the endoscope body. The used scope can then be sterilized or disposed of. A new or sterilized scope can be utilized in a subsequent procedure to prevent contamination between patients.

In many embodiments of the invention, the scope is removably attached to the endoscope body via a connection between the scope connector and body connector, so as to establish an electrical communication between the endoscope body and scope. The connectors may include or be coupled with one or more of various types of electrical connection mechanisms, such as but not limited to wires or other connection mechanisms capable of transferring power and/or data between components of the endoscope body and scope. For example, the electrical connections may allow an image sensor in the scope to pass an image signal to the endoscope body. The electrical connections may also allow electrical power to be passed from the cable through the endoscope body to power the image sensor and an illumination module in the scope. The connectors may also include mechanical connectors, such as but not limited to locking mechanisms like a pin or thread.

In certain embodiments of the invention, the scope includes an identification module that electronically stores an identification number unique to each scope, and/or allows for checking for non-compliant re-use. The identification module can be implemented in various ways, including but not limited to a memory coupled with the scope connector. The endoscope system may include circuitry to count the number of mating cycles between the endoscope body and various scopes, and may include a use limiter to preclude more than a certain number of uses.

The scope may alternatively or additionally be identified by hardware characteristics, such as but not limited to the calibration or pixelation characteristics of the image sensor. As an example, a calibration algorithm may be used as the identification number for the scope.

In some embodiments of the invention, within the scope itself are also electrical connection mechanisms such as but not limited to wires such as wires 128 or other connection mechanisms capable of transferring power and/or data between components of the scope tip at the distal end of the scope, and components at the proximal end of the scope. These connection mechanisms can be housed in an elongated casing such as elongated casing 129.

In some embodiments of the invention, the scope is made of one or more of a variety of materials suitable for immersion in liquid in its entirety, without compromising its effectiveness or safety in performing endoscopy procedures. The materials can include but not limited to one or a combination of rigid, flexible and/or semi-flexible materials, such as but not limited to stainless steel, nickel titanium (e.g., Nitinol), polyvinyl chloride and/or silicone. For example but not by way of limitation, a flexible materials may be reinforced or embedded with a rigid material. The scope may be configured such that it can be placed in chemical disinfectant for a particular minimum amount of time deemed sufficient for proper disinfection. As an example, the scope connector can be sealed so as to allow power and data signals to pass through the scope, but not allow liquids or potentially contaminating substances to enter the scope. This can be done by using potting methods such as epoxy placed around the connecting pins within the connector subassembly. It can also be done by bonding or welding ring-shaped materials around each pin.

Alternatively or additionally, the scope may be sterilized or disinfected using methods such as but not limited to EtO sterilization, Gamma sterilization, or high-level disinfectant immersion. The scope may include components that are resilient and/or hardened against radiation and/or chemicals such as but not limited to ethylene oxide, so as to withstand certain sterilization methods.

In certain embodiments of the invention, the scope is intended to be disposable, with a new, sterile replacement scope being used for each endoscopy procedure. Disposable versions of the detachable scope may include different design decisions than in the reusable, disinfectable versions, to lower manufacturing costs of the scope. For example but not by way of limitation, in a disposable version, less costly polycarbonate material may be utilized instead of sapphire for a window or lens. As another example, a reusable version of the scope may be manufactured with more protection around prongs of various connectors where ingress could otherwise occur.

The scope can have a combination of one or more of various lengths and diameters, configured to be appropriate for its intended use. In certain embodiments of the invention, the scope for use in nasoendoscopy can have a diameter of between 2 mm to 4 mm, and a length of between 12 mm to 127 mm. The scope can have a single diameter or varying diameters along its length. The scope can be flexible, rigid, or a combination thereof, and may or may not include a partial and/or complete casing for the scope components. In certain embodiments of the invention, the ends can be sealed using a sealant, such as but not limited to epoxy, so as to be resistant to entry of fluids and/or other substances. Alternatively and/or additionally, components can be press-fit into a machined housing, and/or a third material is bonded or welded to both components (such as but not limited to an optic and casing).

While various detachable scopes are described above with respect to FIG. 1, other detachable scopes may be utilized as appropriate to the requirements of a specific application in accordance with various embodiments of the invention. Scope tips in accordance with a number of embodiments of the invention are discussed further below.

### Scope Tips

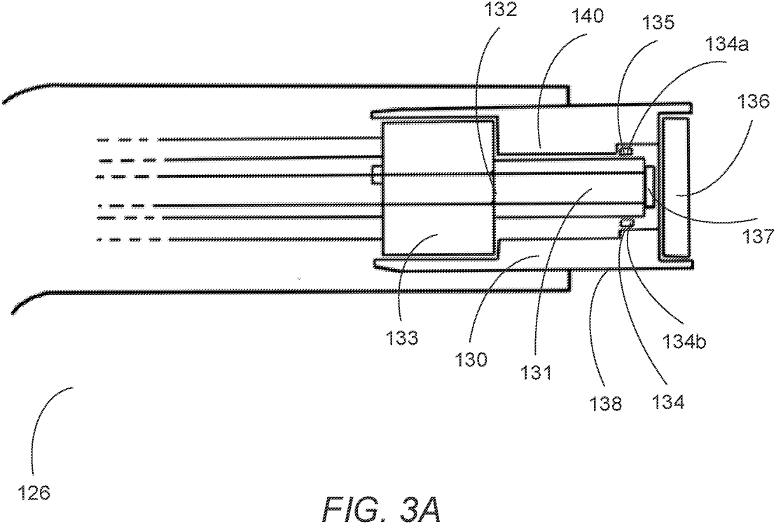

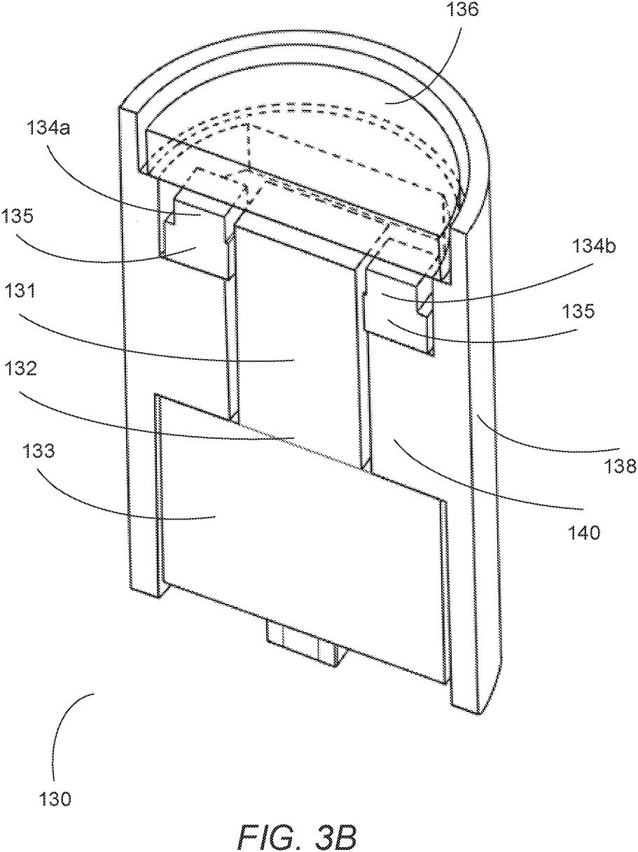

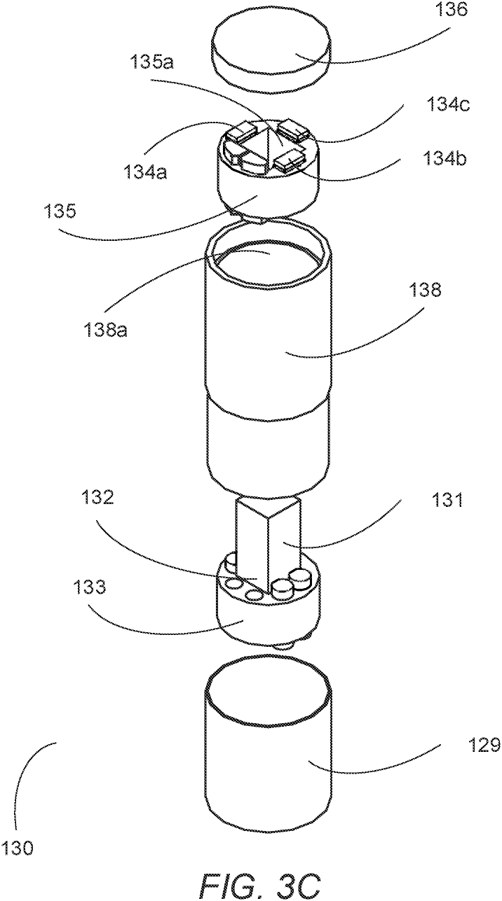

A scope tip, such as scope tip 130, at the distal end of the scope, in accordance with many embodiments of the invention, is shown in further detail in FIGS. 3A-3C and includes various components such as but not limited to an image sensor 132, an illumination module 134, and/or an optic 136.

In some embodiments of the invention, the scope tip may include a tip casing 138, such as but not limited to a hollow spacer tube, in which components of the scope tip are disposed and/or fixed. The tip casing may be constructed out of one or more of various materials safe for direct internal human contact, such as but not limited to a stainless steel alloy or a biocompatible plastic like polyether ether ketone (PEEK). The materials for the tip casing may exhibit low or no thermal and electrical conductivity. The interior of the tip casing and/or optic may be coated with a non-reflective and/or nonconductive material. In some embodiments of the invention, the scope tip is a separate component affixed to the elongated structure of the scope. For certain applications such as but not limited to nasoendoscopy, the scope tip may have an outer diameter of 4 mm or less.

In many embodiments of the invention, the optic, such as but not limited to a lens and/or window, is optically clear and is disposed at the end of the scope tip that enters the patient. The optic may be permanently fixed to tip casing using a permanent fastener, such as but not limited to adhesive or welding. The optic can be made of any suitable material safe for direct internal human contact, and/or that is capable of being disinfected while maintaining its suitability for use in an endoscopy, such as but not limited to sapphire. The optic and/or its adjacent portions of the tip casing may be sealed to resist fluid entry.

In a number of embodiments of the invention, the illumination module is disposed near the optic, so as to illuminate the internal anatomy of the patient visible from the optic. Placing the illumination module relatively close to the optic can improve lighting and reduce reflection. Alternatively, the illumination module can be disposed elsewhere but in such a way as to light the desired portion(s) for examination. The illumination module includes one or more types of light sources, such as but not limited to light-emitting diodes (LEDs) (such as but not limited to one or more of LEDs 134a-c), a laser diode (LD), an ultraviolet (UV) light source, or an infrared (IR) light source, to provide suitable lighting for the appropriate endoscopy procedure. The light sources can vary in type, brightness, and/or color. In some embodiments, these aspects of lighting can be controlled by the user via a computing system to which the endoscope system is connected, or via an input interface on the endoscope system itself.

In certain embodiments of the invention, the illumination module may include circuitry, such as but not limited to an illumination printed circuit board (PCB) 135 to which light sources are coupled or mounted. The illumination PCB may include an opening, such as but not limited to the rectangular hole shown as opening 135a in FIG. 3C, so as to allow at least a portion of a camera module 131 and/or light rays to pass through. As shown in FIG. 3C, according to one embodiment of the invention, the illumination PCB forms a ring shape allowing for LEDs to be placed, for example, in a circular fashion and mounted over illumination PCB. In a number of embodiments of the invention, this configuration can allow for at least three light sources such as LEDs to be disposed within the illumination module, while keeping the outer diameter of the scope tip to within 3 mm, and in some embodiments, within 2 mm.

According to many embodiments of the invention, the image sensor is disposed within the scope tip, and in relation to the illumination module such that the lighting from illumination module provides an appropriate view to the image sensor of the portion of internal anatomy for examination. The image sensor can incorporate one or more of various types of sensor technologies, such as but not limited to charge-coupled devices (CCD) and active-pixel sensors (CMOS sensors). In some embodiments of the invention, the image sensor and/or other electronic components are coupled with circuitry, such as but not limited to by mounting on an image sensor printed circuit board (PCB) 133. In certain embodiments of the invention, the image sensor is disposed within a camera module 131 and away from the lens (such as shown in FIG. 3A with image sensor 132 and lens 137). Other electrical components may also be disposed within the camera module. In some embodiments of the invention, various aspects of the image sensor functionality can be controlled by the user via a computing system to which the endoscope system is connected, or via an input interface on the endoscope system itself.

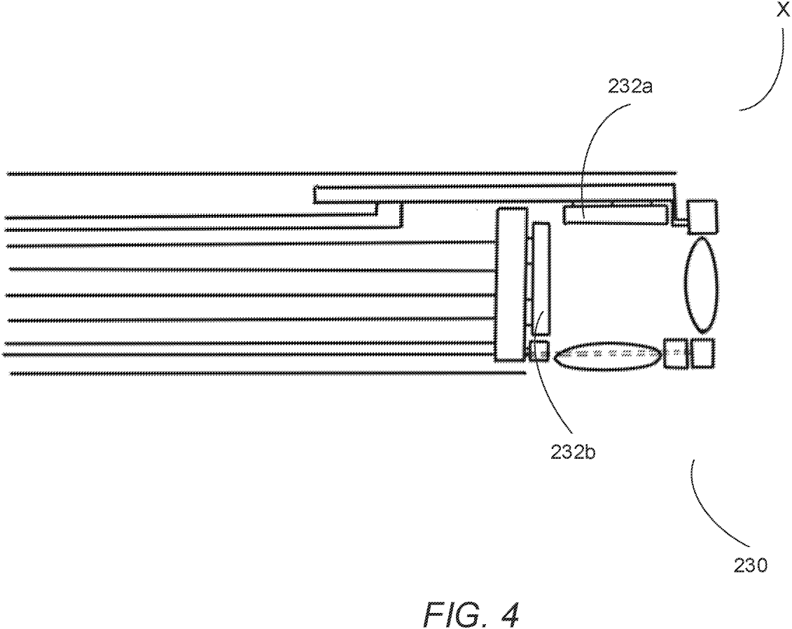

The scope tip can optionally include one or more additional lenses, such as but not limited to lens 137, between the image sensor and the optic. An airgap can be included between an additional lens and the optic . In certain embodiments of the invention, a scope tip such as scope tip 230 includes multiple image sensors facing different directions, such as but not limited to the two image sensors 232a-b shown in FIG. 4. This can allow the user to view different angles and thus different anatomical structures without the need for tip articulation. Alternatively or additionally, the direction of view of one or more of the image sensors can be redirected using optical components such as mirrors or prisms in the scope tip. In a number of embodiments of the invention, an electronically and thermally protective layer 140 is situated between the image sensor and the illumination module. A thermally protective layer can include an airgap, a portion of or the same material as used in the tip casing and/or other material that reduces heat transfer from illumination module to image sensor. Since certain image sensors are sensitive to heat, a thermally protective layer can serve to protect the integrity of the image sensors from heat generated by operation of the illumination module.

In certain embodiments of the invention, the thermally protective layer includes at least a portion of the tip casing which holds the tip components securely in place. This can allow the illumination module to be disposed near the image sensor. Because the thermally protective layer exhibits low- or no electrical and/or thermal conductivity, it prevents circuits in the sensor and illumination module from connecting to each other and thus shorting. This configuration can thus enable the inclusion of two circuit units, such as the sensor and illumination module including their respective PCBs, within a small-diameter scope.

By separating the imaging unit from the illumination module, the two separate circuit units (including, for example, PCBs) could be of a relatively smaller diameter since each circuit unit would only have to accommodate the components and wires for either imaging or illumination. By contrast, for example, a design with an image sensor and LEDs on a single PCB would require a larger-diameter combined unit, and therefore scope tip, to accommodate both sets of components and their respective wires. The larger diameter negatively affects scope dexterity and patient comfort during the procedure. The smaller outer scope diameter (including but not limited to under 4 mm, such as 3 mm or 2 mm) of endoscope systems, according to many embodiments of the invention, can also make them suitable for certain patient populations such as pediatric. Some existing endoscope systems have scope diameters under 4 mm, but they typically have a single PCB module in the tip, thus necessitating an additional means for moving either light into, or image data out of, the patient, usually through fiberoptic means.

In many embodiments of the invention, the tip casing includes an elongated opening (such as but not limited to opening 138a shown in FIG. 3C) in the center through which an image sensor, camera module, and/or light rays can pass. In this opening, the image sensor PCB, illumination PCB and the optic may be held in place through ledges which are machined or injection molded into the tip casing. The components can be press-fit and/or further secured with epoxy to the tip casing.

While various scope tips are described above with respect to FIGS. 3A-3C and 4, other scope tips may be utilized as appropriate to the requirements of a specific application in accordance with various embodiments of the invention. Computing systems in accordance with a number of embodiments of the invention are discussed further below.

### Computing Systems

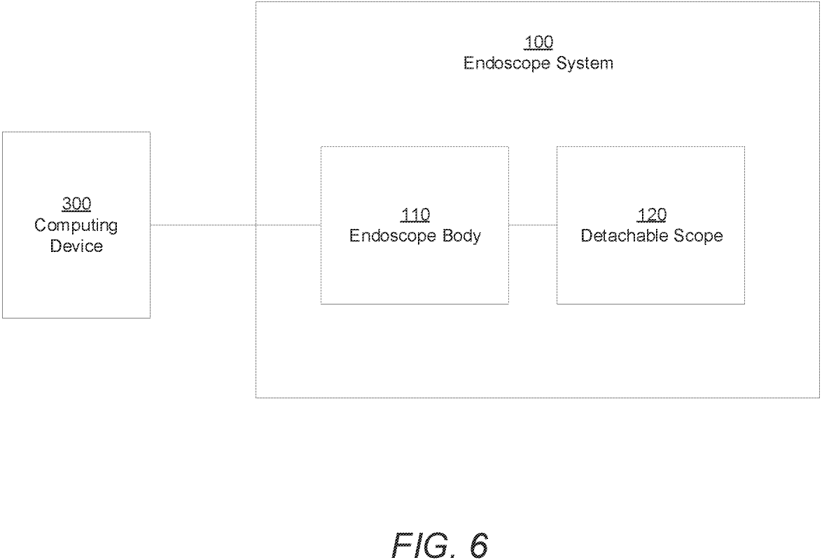

Turning now to FIG. 6, in accordance with some embodiments of the invention, the endoscope system may be connected to at least one computing system 300 via one or more of various wired or wireless connections, such as but not limited to a USB cable. Computing systems may include a display screen and controls for displaying images from a digital image signal onto a display screen. Computing systems can include controls for saving a displayed image. Displayed images can include a single image, a video, or both.

Computing systems may include a personal computer, a laptop computer, a tablet, a smart phone, a monitor and/or any other computing device with sufficient processing power for the processes described herein. A computing system includes a processor, which may refer to one or more devices within the computing system that can be configured to perform computations via machine readable instructions stored within a memory of the computing system. The processor may include one or more microprocessors (CPUs), one or more graphics processing units (GPUs), and/or one or more digital signal processors (DSPs). According to other embodiments of the invention, computing systems may be implemented on multiple computers.

In some embodiments of the invention, computing systems may include an input/output interface that can be utilized to communicate with a variety of devices, including but not limited to a projector, a camera, other display devices and/or the endoscope system. As can be readily appreciated, a variety of software architectures can be utilized to implement a computer system in accordance with several embodiments of the invention.

Although the present invention has been described in certain specific aspects, many additional modifications and variations would be apparent to those skilled in the art. It is therefore to be understood that the present invention can be practiced otherwise than specifically described without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. An endoscope system, comprising:
an endoscope body, comprising:
a cable capable of transmitting image data to a computing system; and
a body connector; and
a detachable scope, comprising:
a scope connector configured to couple with the body connector; and
a tip, comprising:
an image sensor, wherein image data produced by the image sensor is transmitted to the endoscope body via the scope connector; and
an illumination module capable of providing light.

2. The endoscope system of claim 1, wherein the illumination module is physically separated from the image sensor such that the image sensor is protected from heat generated by operation of the illumination module.

3. The endoscope system of claim 1, wherein the image sensor is coupled with an image sensor printed circuit board (PCB) and the illumination module includes an illumination printed circuit board (PCB), and wherein the tip has a diameter of less than 3 mm.

4. The endoscope system of claim 1, wherein the detachable scope is configured to be resistant to liquid in its entirety such that when detached from the endoscope body, the detachable scope can be immersed in its entirety in liquid disinfectant while maintaining suitability for use in an endoscopy procedure subsequent to the immersion.

5. The endoscope system of claim 1, wherein the endoscope body further comprises:
a main circuitry coupled with the cable for receiving power from the computing system.

6. The endoscope system of claim 1, wherein the endoscope body further comprises:
an image processor coupled with the body connector to receive the image data, wherein the image processor is capable of processing the image data and transmitting the processed image data to the computing system.

7. The endoscope system of claim 1, wherein the cable is a Universal Serial Bus (USB) cable.

8. The endoscope system of claim 1, wherein the computing system includes at least one device selected from the group consisting of a personal computer, a monitor, a tablet and a smartphone.

9. A detachable scope for an endoscope system, comprising:
a scope connector configured to couple with a body connector of an endoscope body; and
a tip, comprising:
an illumination module capable of providing light; and
an image sensor, wherein image data produced by the image sensor is transmitted to the endoscope body via the body connector.

10. The detachable scope of claim 9, wherein the illumination module is physically separated from the image sensor such that the image sensor is protected from heat generated by operation of the illumination module.

11. The endoscope system of claim 9, wherein the image sensor is coupled with an image sensor printed circuit board (PCB) and the illumination module includes an illumination printed circuit board (PCB), and wherein the tip has a diameter of less than 3 mm.

12. The detachable scope of claim 9, wherein the detachable scope is configured to be resistant to liquid in its entirety such that when detached from the endoscope body, the detachable scope can be immersed in its entirety in liquid disinfectant while maintaining suitability for use in an endoscopy procedure subsequent to the immersion.

13. The detachable scope of claim 9, wherein the detachable scope is of an elongated shape and is coupled to the endoscope body such that a proximal end of the elongated shape is disposed near the endoscope body and a distal end of the elongated shape extends away from the endoscope body.

14. An endoscope body for an endoscope system, comprising:
a cable capable of transmitting image data to a computing system; and
a body connector configured to couple with a scope connector of a detachable scope, wherein image data produced by an image sensor of the detachable scope is received from the detachable scope via the body connector.

15. The endoscope body of claim 14, wherein the endoscope body further comprises:
a main circuitry coupled with the cable for receiving power from the computing system.

16. The endoscope body of claim 14, further comprising:
an image processor coupled with the body connector to receive the image data, wherein the image processor is capable of processing the image data and transmitting the processed image data to the computing system.

17. The endoscope body of claim 14, wherein the cable is a Universal Serial Bus (USB) cable.

18. The endoscope body of claim 14, wherein the computing system includes at least one device selected from the group consisting of a personal computer, a monitor, a tablet and a smartphone.

19. The endoscope body of claim 14, wherein at least one portion of the endoscope body is formed in the shape of a handle.

20. An endoscope system, comprising:
an endoscope body, comprising:
a USB cable capable of transmitting image data to a computing system, wherein upon coupling the cable with the computing system, the endoscope system is automatically recognized as a camera device by the computing system; and
a body connector; and
a detachable scope, comprising:
a scope connector configured to couple with the body connector; and
a tip, comprising:
an image sensor coupled with an image sensor printed circuit board (PCB), wherein image data produced by the image sensor is transmitted to the endoscope body via the scope connector; and
an illumination module including an illumination printed circuit board (PCB) coupled with three light-emitting diodes (LEDs),
wherein the illumination module is physically separated from the image sensor such that the image sensor is protected from heat generated by operation of the illumination module, wherein the image sensor PCB and the illumination PCB are configured so as to enable the tip to have a diameter of less than 3 mm.
